(19) United States
(12) Patent Application Publication
(10) Pub. No.: US 2006/0167386 A1
(43) Pub. Date: Jul. 27, 2006

(54) Blood vessel graft sizer

(76) Inventors: Daniel H. Drake, Traverse City, MI (US); Daniel K. Farley, Traverse City, MI (US); Anthony J. Mulac, Traverse City, MI (US)

Correspondence Address: MCANDREWS HELD & MALLOY, LTD, 500 WEST MADISON STREET, SUITE 3400, CHICAGO, IL, 60661, US

(21) Appl. No.: 11/041,544

(22) Filed: Jan. 24, 2005

(51) Int. Cl. A61B 5/103; G01B 1/00

(52) U.S. Cl. 600/587; 33/512

(57) ABSTRACT

A vascular graft sizer for gauging the size of a distorted blood vessel includes a sizing member. The sizing member has a shape substantially similar to a shape of a distorted blood vessel, and corresponds to a vascular graft that has a shape substantially similar to a non-distorted blood vessel.

### CROSS-REFERENCE TO RELATED APPLICATION

None.

### STATEMENT REGARDING FEDERALLY SPONSORED RESEARCH OR DEVELOPMENT

None.

### BACKGROUND OF THE INVENTION

The present invention relates to vascular graft sizers for determining an appropriate vascular graft to use during surgical replacement of a portion of a blood vessel.

Disease and atrophy of a patient's arterial system can result in an aneurysm, or the ballooning of a blood vessel. FIG. 1 illustrates an example of an aneurysm, specifically, an abdominal aortic aneurysm. The abdominal aorta is a portion of the main blood vessel carrying oxygenated blood from the left ventricle of the heart to the rest of the body. Aneurysms present a risk of, among other things, rupture and internal bleeding, which can be fatal. To reduce that risk, surgical treatment of aneurysms is sometimes necessary.

Sometimes the most diseased portion of the blood vessel must be removed due to an aneurysm and replaced with a vascular graft, which may be synthetic. For the replacement of a portion of the abdominal aorta, grafts are generally circular to match the original cross-section of the blood vessel. One example of a material used for such grafts is a flexible tube of Gortex. Because end to end anastomosis (sewing, suturing) of the graft to the portions of the original blood vessel left intact is required, the cross section of the graft must match the cross section of the blood vessel.

Before the graft is implanted, a portion of the blood vessel affected by the aneurysm is removed. To accomplish this, the blood vessel is first cross-clamped at a healthy section near the diseased section to prevent any blood flow through the diseased area while the vessel is being repaired. Then, the diseased section of the aorta is removed, allowing the diameter of the healthy section of the vessel to shrink back approximately to its original diameter. The surgeon often reinforces the open ends of the vessel with felt to facilitate suturing of the graft to the vessel.

At this point, the surgeon may determine the desired diameter for the graft. Earlier measurement of the vessel to determine an appropriate graft size is not possible because of the ballooning of the diseased portion prior to removal, and also due to the addition of the felt. The graft diameter is typically measured in millimeters, and generally varies between 22-34 millimeters.

The graft size to be used may be determined by the use of a sizer. Current sizers are round or half-round devices that are slipped on to an open end of the blood vessel and compared with the blood vessel to determine an appropriate match. Typical current sizers match the profile of the graft to be used and the profile of an undistorted blood vessel. However, because the blood vessel is clamped, often approximately 1 cm from the portion of the blood vessel to which the graft will be added, the open end of the blood vessel is not round, but instead distorted from its original shape. FIG. 2 illustrates an example of distortion of a blood vessel near a clamp. This distortion from the generally circular shape of the blood vessel forces a surgeon to use some guesswork to determine the graft size when comparing the blood vessel to a half-round sizer. In many cases, the first guess based on the use of a current sizer is incorrect. Each incorrectly sized graft attempted to be used must be disposed of, until the correct size is found. This increases the amount of time of the surgery as well as the cost, as the grafts are expensive. Additionally, if the graft is sized correctly at one end but is a serious mismatch at the other, potentially fatal complications from attempting repair can occur.

It is therefore one object of the present invention to eliminate one or all of the problems associated with known vascular graft sizers, including improving the accuracy of measuring a clamped blood vessel and reducing or eliminating the amount of guesswork in determining an appropriate graft size.

### BRIEF SUMMARY OF THE INVENTION

A vascular graft sizer for gauging the size of a distorted blood vessel is disclosed that includes a sizing member. The sizing member has a shape substantially similar to a shape of a distorted blood vessel. Further, the sizing member corresponds to a vascular graft that has a shape substantially similar to a non-distorted blood vessel. For example, a substantially oval shape, a substantially oblong shape, or a substantially elliptical shape may be used for the shape of the sizing member. Additionally, the vascular graft sizer may include a graft size indicator proximal to the sizing member for indicating the size of a vascular graft to which the sizing member corresponds.

The vascular graft sizer may also include a handle portion and a second sizing member. The handle portion is interposed between the sizing member and the second sizing member. The sizing member and the second sizing member both correspond to a common vascular graft of a given size. The second sizing member has a shape similar to the shape of the sizing member, but the shapes of the sizing member and the second sizing member may have at least one different dimension.

A vascular graft sizing system for gauging the size of a distorted blood vessel is disclosed that includes a first sizing member, a second sizing member, and a handle portion. The first sizing member includes a first face that has a first shape that corresponds to a shape of a distorted blood vessel. The first sizing member corresponds to a vascular graft that has a shape substantially similar to a non-distorted blood vessel. The second sizing member includes a second face that has a second shape that corresponds to a shape of a distorted blood vessel. The second sizing member corresponds to a vascular graft that has a shape substantially similar to a non-distorted blood vessel. The handle portion is interposed between the first and second sizing members.

The first face may include two round portions joined by flat portions. The first face may also include a width defined by the distance between the flat portions. Similarly, the second face may include two round portions joined by flat portions, with a width defined by the distance between the flat portions. The first and second sizing members may correspond to a common vascular graft, and their respective widths are preferably different.

Further, the handle portion may include a handle interposed between two stems. One of the stems is located proximal to the first sizing member, and the other stem is located proximal to the second sizing member. The two stems are flexibly adjustable.

### BRIEF DESCRIPTION OF THE SEVERAL VIEWS OF THE DRAWINGS

FIG. 1 is a view of an aorta with an aneurysm.

FIG. 2 is a view of an aorta with a section removed and an open end clamped showing distortion of the clamped open end of the aorta.

FIG. 3 is a side view of a vascular graft sizer formed in accordance with an embodiment of the present invention.

FIG. 4 is an end view of the embodiment illustrated in FIG. 3 as seen from View 4-4.

FIG. 5 is an end view of the embodiment illustrated in FIG. 3 as seen from View 5-5.

FIG. 6 illustrates a top view of a vascular graft sizing member formed in accordance with another embodiment of the present invention.

FIG. 7 is an end view of a vascular graft sizer formed in accordance with another embodiment of the present invention.

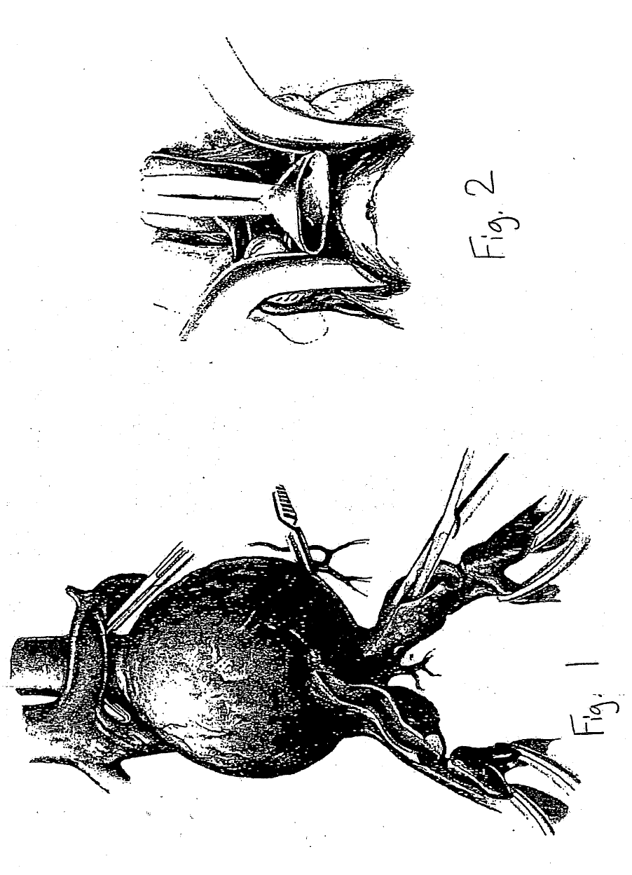

### DETAILED DESCRIPTION

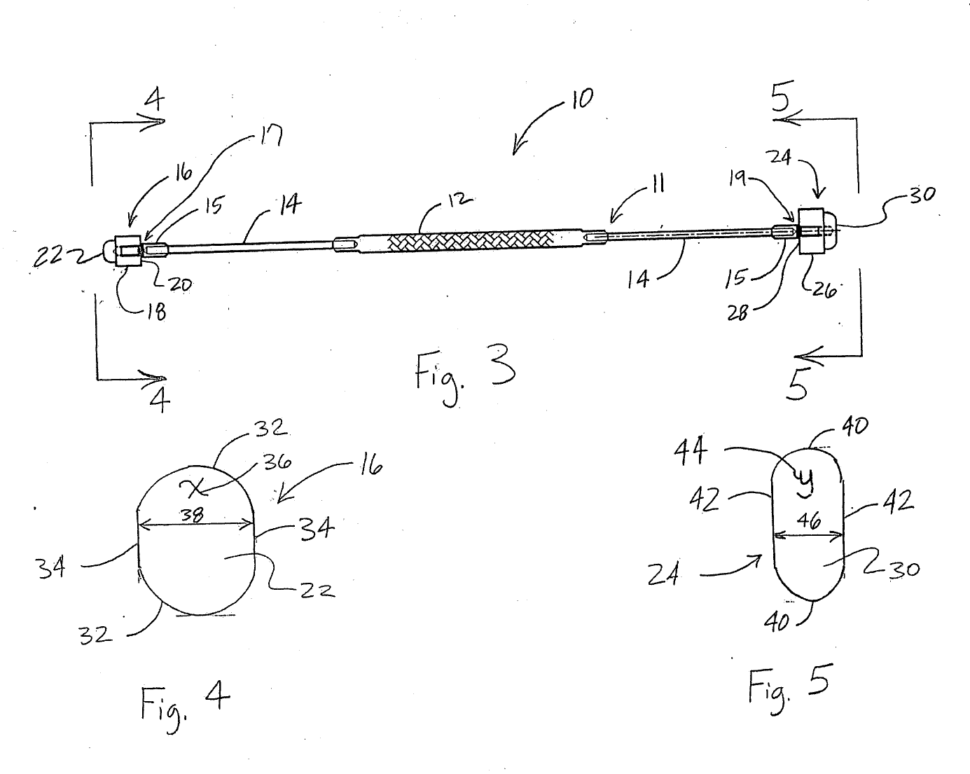

FIGS. 3 through 5 present different views of one embodiment of the present invention. FIG. 3 provides a side view of a vascular graft sizer 10. FIGS. 4 and 5 show end views of the vascular graft sizer 10 taken from View 4-4 and View 5-5, respectively.

As shown in FIG. 3, the vascular graft sizer 10 includes a handle portion 11, adapters 15, a first sizing member 16, and a second sizing member 24. The handle portion 11 is interposed between the first sizing member 16 and the second sizing member 24. The handle portion 11 may include a handle 12 and stems 14. The handle 12 may be made of plastic, and the stems 14 may be made of a flexible metal. The stems 14 may be joined to the handle 12 by, for example, a press fit. One of the stems 14 extends from the handle 12 toward the first sizing member 16 and joins an adapter 15 by, for example, a press fit. The adapter 15 includes a threaded end that threads into a threaded hole 17 of the first sizing member 16. Thus, the adapter 15 facilitates the joining of the handle portion 11 to the first sizing member 16. Another stem 14 extends from the opposite side of the handle 12 toward the second sizing member 24 and joins the handle portion 11 to the second sizing member 24 via an adapter 15. Preferably, the handle 12 has a low profile to permit greater visibility during the use of the vascular graft sizer 10. In the illustrated embodiment, the stems 14 are flexibly adjustable to provide for easier handling and positioning of the vascular graft sizer 10. To change to differently sized sizing members, the first sizing member 16 and/or the second sizing member 24 are rotated to disengage the threads of the adapter 15, and the first sizing member 16 and/or the second sizing member 24 are removed. A different sizing member is then attached at the threaded end of an adapter 15. This allows the handle portion 11 to be used with many sizing members. Alternatively, quick-release mechanisms commonly used in medical devices (for example, as disclosed in U.S. Pat. No. 5,984,865, incorporated herein by reference) could be incorporated to temporarily join the handle portion 11 to the first sizing member 16 and second sizing member 24 to allow one handle portion 11 to be used with many sizing members.

The first sizing member 16 of the vascular graft sizer 10 includes a first face 22 and a first rearward surface 20 joined by a first side 18. The first face 22 has an oblong shape, shown in FIG. 4. The first sizing member 16 includes a threaded hole 17 extending into the first rearward surface 20. A stem 14 of the handle portion 11 is joined to the first sizing member 16 proximal to the first rearward surface 20 by an adapter 15 that threads into the threaded hole 17. The second sizing member 24 of the vascular graft sizer 10 includes a second face 30 and a second rearward surface 28 joined by a second side 26. The second face 30 has an oblong shape generally similar to the oblong shape of the first face 22, shown in FIG. 5. The second sizing member 24 includes a threaded hole 19 extending into the second rearward surface 28. A stem 14 of the handle portion 11 is joined to the second sizing member 24 proximal to the second rearward surface 28 by an adapter 15 that threads into the threaded hole 19.

FIG. 4 illustrates an end view of the vascular graft sizer 10 from FIG. 3 as seen from View 4-4. As seen in FIG. 4, the first sizing member 16 of the vascular graft sizer 10 includes two round portions 32 joined by two flat portions 34. The distance between the flat portions 34 defines a width 38. Additionally, the first face 22 may include a graft size indicator 36. The graft size indicator 36 gives a visual indication of the graft size to which the first sizing member 16 corresponds. Preferably, the graft size indicator 36 (represented in FIG. 4 as an “x”) is a number corresponding to the diameter of the corresponding graft size in millimeters.

FIG. 5 illustrates an end view of the vascular graft sizer 10 from FIG. 3 as seen from View 5-5. As seen in FIG. 5, the second sizing member 24 of the vascular graft sizer 10 includes two round portions 40 joined by two flat portions 42. The distance between the flat portions 42 defines a width 46. The width 46 differs from the width 38 (FIG. 4). Additionally, the second face 30 may include a graft size indicator 44. The graft size indicator 44 gives a visual indication of the graft size to which the second sizing member 24 corresponds. Preferably, the graft size indicator 44 (represented in FIG. 5 as a “y”) is a number corresponding to the diameter of the corresponding graft size in millimeters. The indicators 36 (“x”) and 44 (“y”) may be the same, indicating that each sizing member 16, 24 corresponds to the same size graft, or different, indicating that the sizing members 16, 24 correspond to different sized grafts.

The first sizing member 16 and second sizing member 24 may be made of, for example, stainless steel, aluminum, titanium, or plastic. Preferably, the first sizing member 16 and second sizing member 24 are made of a clear plastic, allowing increased visibility to a surgeon during their use.

In the embodiment illustrated in FIGS. 3-5, both the first sizing member 16 and second sizing member 24 may correspond to the same size vascular graft, though they have a different dimension (for example, from above, the width 38 is different from the width 46). This allows a surgeon a greater variety of sizes to accommodate different shapes of distorted blood vessels, which can vary based on, among other reasons, the location of a clamp on the blood vessel during a specific procedure. As another example, some embodiments of the present invention may have first and second sizing members that have profiles substantially different from each other. By having sizing members of different dimensions and/or profiles available, a more accurate approximation of graft size may be made for different shapes of distorted blood vessels, which may vary from procedure to procedure.

In practice, the vascular graft sizer 10 may be used as part of a set including several sizers and/or sizing members covering a range of available vascular graft sizes. After removing the desired portion of the blood vessel, and with the blood vessel still clamped, the surgeon places various sizing members into the distorted open end of the clamped blood vessel until finding an appropriate match. Next, a vascular graft corresponding to the appropriately selected vascular graft sizer is compared to the blood vessel to verify that the size is correct. By using a shape or shapes that more closely match the distorted blood vessel instead of the corresponding graft (and undistorted blood vessel), the vascular graft sizer 10 provides a more accurate estimation of blood vessel diameter, thereby eliminating or reducing incorrect graft size estimates, and reducing the time required to surgically repair the blood vessel, and also reducing or eliminating the cost of attempting to use improperly sized grafts which must be thrown away after attempted use.

FIG. 6 illustrates a top view of a vascular graft sizing member 50 formed in accordance with another embodiment of the present invention. The vascular graft sizing member 50 includes a threaded hole 52 that accepts an adaptor and/or a portion of a handle similar to the above described embodiment, thereby allowing the manipulation and positioning of the sizing member 50 for determining the size of a blood vessel, as well as allowing interchangeability of differently sized sizing members.

The sizing member 50 includes a rearward surface 56 and a face 58 joined by a side 54. The sizing member 50 includes a leading edge 62 formed at or near the intersection of the face 58 and the side 60. The face 58 is shaped to correspond to the shape of the open end of a blood vessel distorted by clamping. For example, the face 58 may be oblong, elliptical, or oval. The threaded hole 52 extends into the sizing member 50 from the rearward surface 56. The sizing member 50 also includes an introducing portion 64 that extends from the face 58.

The introducing portion 64 includes a tapering surface 66 and a leading face 68. In the illustrated embodiment, the tapering surface 66 is curved. The leading face 68 has a smaller area than the face 58, and the tapering surface 66 tapers inwardly from the leading edge 62 to the leading face 68. The side 54 includes grooves 60 along the outer perimeter of the sizing member 50. The grooves 60 provide a visual cue that it is the outside of the side 54 that corresponds to the blood vessel and graft being used. When placed in a blood vessel, the sizing member 50 is introduced with the leading face 68 entering the open end of the blood vessel first. Because the leading face 68 has a smaller area than the face 58, the introducing portion 64 makes the sizing member 50 easier to insert into a blood vessel during the use of the sizing member 50. The grooves 60 are inside the blood vessel being sized after the sizing member 50 is completely inserted.

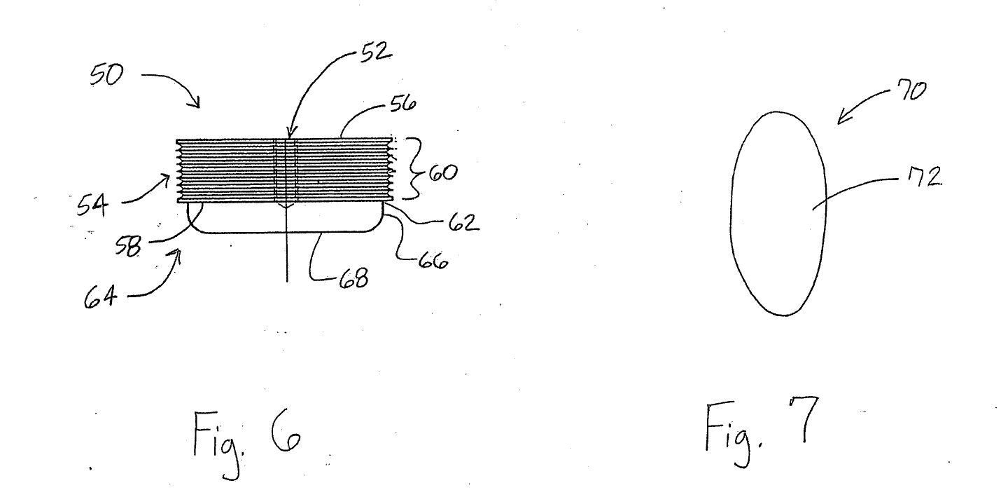

FIG. 7 illustrates an end view of a vascular graft sizer 70 formed in accordance with another embodiment of the present invention. The vascular graft sizer 70 includes a face 72 that has the shape of an ellipse. Depending on the procedure and vascular graft to be used, other shapes differing from the generally circular cross-section of an undistorted blood vessel may also be used.

While particular embodiments of the invention have been shown, it will be understood that the invention is not limited thereto since modifications may be made by those skilled in the art, particularly in light of the foregoing teaching. It is therefore, the appended claims that define the true spirit and scope of the invention.

What is claimed is:

1. A vascular graft sizer for gauging the size of a distorted blood vessel comprising a sizing member, said sizing member having a first shape substantially similar to a shape of a distorted blood vessel, said sizing member corresponding to a vascular graft having a shape substantially similar to a non-distorted blood vessel.

2. The vascular graft sizer of claim 1 wherein said first shape is substantially oval.

3. The vascular graft sizer of claim 1 wherein said first shape is substantially elliptical.

4. The vascular graft sizer of claim 1 further comprising a handle portion and a second sizing member, said handle portion interposed between said sizing member and said second sizing member, said sizing member and said second sizing member both corresponding to a common vascular graft having a given size, said second sizing member having a second shape having a similar profile to said first shape but having a different dimension.

5. The vascular graft sizer of claim 1 further comprising a graft size indicator proximal to said sizing member for indicating the size of a vascular graft to which said sizing member corresponds.

6. A vascular graft sizer for gauging the size of a distorted blood vessel comprising an oblong sizing member, said oblong sizing member corresponding to a vascular graft having a substantially circular shape.

7. The vascular graft sizer of claim 6 wherein said oblong sizing member is substantially oval.

8. The vascular graft sizer of claim 6 wherein said oblong sizing member is substantially elliptical.

9. The vascular graft sizer of claim 6 further comprising an introducing portion adapted to ease entry of said oblong sizing member into a blood vessel, said oblong sizing member comprising a face, and said introducing portion extending from said face of said oblong sizing member.

10. The vascular graft sizer of claim 6 further comprising a handle portion and a second oblong sizing member, said handle portion interposed between said oblong sizing member and said second oblong sizing member, said oblong sizing member and said second oblong sizing member both corresponding to a common vascular graft having a given size, said second oblong sizing member having a similar profile to said oblong sizing member but having a different dimension.

11. The vascular graft sizer of claim 10 wherein said oblong sizing member has a first face that is substantially elliptical in shape and has a first width, said second oblong sizing member having a second face that is substantially elliptical in shape and has a second width, and said first width and said second width are different.

12. A vascular graft sizing system for gauging the size of a distorted blood vessel comprising
a first sizing member, said first sizing member including a first face having a first shape corresponding to a shape of a distorted blood vessel, said first sizing member corresponding to a vascular graft having a shape substantially similar to a non-distorted blood vessel;
a second sizing member, said second sizing member including a second face having a second shape corresponding to a shape of a distorted blood vessel, said second sizing member corresponding to a vascular graft having a shape substantially similar to a non-distorted blood vessel; and
a handle portion interposed between said first and second sizing members.

13. The vascular graft sizing system of claim 12 wherein at least one of said first and second faces are substantially oval in shape.

14. The vascular graft sizing system of claim 12 wherein at least one of said first and second faces are substantially elliptical in shape.

15. The vascular graft sizing system of claim 12 wherein said first and second sizing members correspond to a common vascular graft.

16. The vascular graft sizing system of claim 15 wherein said first and second faces have substantially similar profiles but have a different dimension.

17. The vascular graft sizing system of claim 12 wherein
said first face includes two first round portions joined by two first flat portions, said first face including a first width defined by the distance between said two first flat portions;
said second face includes two second round portions joined by two second flat portions, said second face including a second width defined by the distance between said two second flat portions;
said first and second sizing members correspond to a common vascular graft; and
said first and second widths are different.

18. The vascular graft sizing system of claim 12 wherein at least one of said first and second sizing members includes a graft size indicator for indicating the size of a vascular graft to which said first or second sizing member corresponds.

19. The vascular graft sizing system of claim 12 wherein said handle portion includes a handle interposed between two stems, one of said two stems is located proximal to said first sizing member and the other of said two stems is located proximal to said second sizing member, and said two stems are flexibly adjustable.

20. The vascular graft sizing system of claim 12 wherein at least one of said first and second sizing members includes an introducing flange adapted to ease entry of said first or second sizing member into a blood vessel, said introducing flange extending from said first or second face of said first or second sizing members, respectively.
